(19) United States
(12) Patent Application Publication
(10) Pub. No.: US 2019/0125527 A1
(43) Pub. Date: May 2, 2019

(54) SYNTHETIC PROSTHETIC VALVE LEAFLET

(71) Applicant: W. L. Gore & Associates, Inc., Newark, DE (US)

(72) Inventors: Valerie R. Binetti, Flagstaff, AZ (US); Karl Busalacchi, Flagstaff, AZ (US); Cody L. Hartman, Flagstaff, AZ (US); Jack J. Hegenbarth, Wilmington, DE (US); Roy Manygoats, JR., Flagstaff, AZ (US); Rachel Radspinner, Flagstaff, AZ (US); Justin A. Swain, Oxford, PA (US)

(21) Appl. No.: 16/129,591

(22) Filed: Sep. 12, 2018

Related U.S. Application Data
(60) Provisional application No. 62/579,783, filed on Oct. 31, 2017.

(51) Int. Cl. A61F 2/24; A61L 27/56

(52) U.S. Cl. CPC A61F 2/2418; A61F 2/2415; A61L 27/56; A61L 27/28; A61F 2/2427; A61F 2230/0069; A61F 2220/0075

(57) ABSTRACT

Thin, biocompatible, high-strength, composite materials are disclosed that are suitable for use in medical devices, such as a prosthetic valve for regulating blood flow direction. In one aspect, the leaflet material maintains flexibility in high-cycle flexural applications, making it particularly applicable to high-flex implants such as a prosthetic heart valve leaflet. The leaflet material includes a coating of a non-elastomeric TFE-PMVE copolymer.

### CROSS REFERENCE RELATED APPLICATIONS

This patent application claims priority to and the benefit of Provisional Patent Application Ser. No. 62/579,783, entitled LEAFLET, filed Oct. 31, 2017, which is incorporated by reference herein in its entirety.

### FIELD

The materials disclosed relate to materials used in medical implants/devices and medical devices incorporating the materials. More particularly, a biocompatible material suitable for use in high-cycle flexural applications including prosthetic valves.

### BACKGROUND

Medical devices, including synthetic polymer prosthetic valve leaflets should exhibit sufficient durability for at least four hundred million pulsatile cycles under representative cardiovascular conditions. The leaflet, for example, must resist structural degradation including the formation of holes, tears, and the like as well as adverse biological consequences including calcification and thrombosis.

A variety of polymeric materials has previously been employed as prosthetic heart valve leaflets. During the cardiac cycle, a prosthetic valve leaflet is subjected to a range of stresses arising from bending. Particular portions of the leaflet are exposed to bending that can result in splits or voids that form in the leaflet creating a site into which blood elements can penetrate. Blebs of fluid, or even thrombus, can affect leaflet motion, can calcify, can affect valve function, and ultimately lead to premature valve failure.

There is a continued need in the art to address the means to improve prosthetic valve leaflets.

### BRIEF DESCRIPTION OF THE DRAWINGS

The accompanying drawings are included to provide a further understanding of this disclosure and are incorporated in and constitute a part of this specification, illustrate embodiments of this disclosure, and together with the description serve to explain the principles of the disclosure.

FIG. 1 is a perspective view of a prosthetic valve in accordance with an embodiment;

FIG. 2 is a cross sectional view of a prosthetic valve leaflet in accordance with an embodiment;

FIG. 3 is a cross sectional view of a prosthetic valve leaflet in accordance with another embodiment;

FIG. 4A is a scanning electron micrograph image of expanded fluoropolymer membrane used to form valve leaflets, in accordance with an embodiment;

FIG. 4B is a scanning electron micrograph image of expanded fluoropolymer membrane used to form valve leaflets, in accordance with an embodiment;

FIG. 4C is a scanning electron micrograph image of expanded fluoropolymer membrane used to form valve leaflets, in accordance with an embodiment;

FIG. 5A is a scanning electron micrograph image of the surface of microporous polyethylene membrane used to form valve leaflets, in accordance with an embodiment;

FIG. 5B is a scanning electron micrograph image of a cross-section of the microporous polyethylene membrane of FIG. 5A, in accordance with an embodiment;

FIG. 6A is a scanning electron micrograph image of stretched microporous polyethylene membrane used to form valve leaflets, in accordance with an embodiment;

FIG. 6B is a scanning electron micrograph image of a cross-section of the microporous polyethylene membrane of FIG. 6A, in accordance with an embodiment; and

FIG. 7 is a plot of PMVE wt % vs. Tack Test for various TFE-PMVE compositions, in accordance with embodiments.

### DETAILED DESCRIPTION

References will now be made to embodiments illustrated in the drawings and specific language which will be used to describe the same. It will nevertheless be understood that no limitation of the scope of the embodiments of this disclosure is thereby intended, such alterations and further modifications in the illustrated methods and apparatus, as such further applications of the principles of the disclosure as illustrated therein as being contemplated as would normally occur to one skilled in the art to which the disclosure relates.

Herein, “comprising” encompasses the terms “consisting of” and “consisting essentially of”. The compositions and methods/processes of the present disclosure can comprise, consist of, and consist essentially of the essential elements and limitations of the disclosure described herein, as well as any of the additional or optional ingredients, components, steps, or limitations described herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “membrane” as used herein refers to a porous sheet of material comprising a single composition, such as, but not limited to, expanded fluoropolymer.

The term “leaflet” as used herein in the context of prosthetic valves refers to a component of a one-way valve wherein the leaflet is operable to move between an open and closed position under the influence of a pressure differential. In an open position, the leaflet allows blood to flow through the valve. In a closed position, the leaflet substantially blocks retrograde flow through the valve. In embodiments comprising multiple leaflets, each leaflet cooperates with at least one neighboring leaflet to block retrograde flow of blood. Leaflets in accordance with embodiments provided herein comprise one or more layers of a composite. Leaflets in accordance with embodiments provided herein may have a thickness of less than 350 μm, and in other embodiments, the leaflet has a thickness between 20-65 μm.

The terms “frame” and “support structure” are used interchangeably to refer to an element to which a leaflet is coupled or supported so as to be operable as a prosthetic valve. The support structure may be, but not limited to, stents and conduits.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material.

As used herein, the term “layer” refers to a continuous material as opposed to a discontinuous material such as power and fibers, unless stated otherwise in the description. As used herein, the term “coating” refers to a continuous material as opposed to a discontinuous material such as power and fibers, unless stated otherwise in the description.

The present disclosure addresses a long-felt need for a material that meets the durability and biocompatibility requirements of high-cycle flexural implant applications, such as prosthetic synthetic heart valve leaflets. In accordance with embodiments herein, the leaflet comprises a composite material having at least one porous synthetic polymer membrane layer having a plurality of pores and/or spaces and an elastomer and/or an elastomeric material and/or a non-elastomeric material filling the pores and/or spaces of the at least one synthetic polymer membrane layer. In accordance with other examples, the leaflet further comprises a layer of an elastomer and/or an elastomeric material and/or a non-elastomeric material on the composite material. In accordance with some embodiments, the elastomer and/or an elastomeric material and/or a non-elastomeric material is imbibed with the expanded fluoropolymer membrane such that the elastomer and/or the elastomeric material and/or the non-elastomeric material occupies substantially all of the void space or pores within the expanded fluoropolymer membrane. In accordance with examples, the composite material comprises porous synthetic polymer membrane by weight in a range of about 10% to 90%.

An example of a porous synthetic polymer membrane includes expanded fluoropolymer membrane having a node and fibril structure defining the pores and/or spaces. In some embodiments, the expanded fluoropolymer membrane is expanded polytetrafluoroethylene (ePTFE) membrane. Another example of porous synthetic polymer membrane includes microporous polyethylene membrane.

Examples of an elastomer and/or an elastomeric material and/or a non-elastomeric material include, but are not limited to, copolymers of tetrafluoroethylene and perfluoromethyl vinyl ether (TFE/PMVE copolymer), (per)fluoroalkylvinylethers (PAVE), urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

In some examples, the TFE/PMVE copolymer is an elastomer comprising between 60 and 20 weight percent tetrafluoroethylene and respectively between 40 and 80 weight percent perfluoromethyl vinyl ether. In some examples, the TFE/PMVE copolymer is an elastomeric material comprising between 67 and 61 weight percent tetrafluoroethylene and respectively between 33 and 39 weight percent perfluoromethyl vinyl ether. In some examples, the TFE/PMVE copolymer is a non-elastomeric material comprising between 73 and 68 weight percent tetrafluoroethylene and respectively between 27 and 32 weight percent perfluoromethyl vinyl ether. In some examples, the leaflet is an expanded polytetrafluoroethylene (ePTFE) membrane having been imbibed with TFE-PMVE copolymer comprising from about 60 to about 20 weight percent tetrafluoroethylene and respectively from about 40 to about 80 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces. In some examples, the leaflet is an expanded polytetrafluoroethylene (ePTFE) membrane having been imbibed with TFE-PMVE copolymer comprising from about 67 to about 61 weight percent tetrafluoroethylene and respectively from about 33 to about 39 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces. In some examples, the leaflet is an expanded polytetrafluoroethylene (ePTFE) membrane having been imbibed with TFE-PMVE copolymer comprising from about 27 to about 32 weight percent perfluoromethyl vinyl ether and respectively about 73 to about 68 weight percent tetrafluoroethylene on the blood-contacting surfaces. In some examples, the leaflet is an expanded polytetrafluoroethylene (ePTFE) membrane having been imbibed with TFE-PMVE copolymer comprising from about 73 to about 68 weight percent tetrafluoroethylene and respectively about 27 to about 32 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces on the blood-contacting surfaces.

The TFE and PMVE components of the TFE-PMVE copolymer are presented herein in weight percent (wt %). For reference, the wt % of PMVE of about 40, 33-39, and 27-32 corresponds to a mole percent (mol %) of about 29, 23-28, and 18-22, respectively.

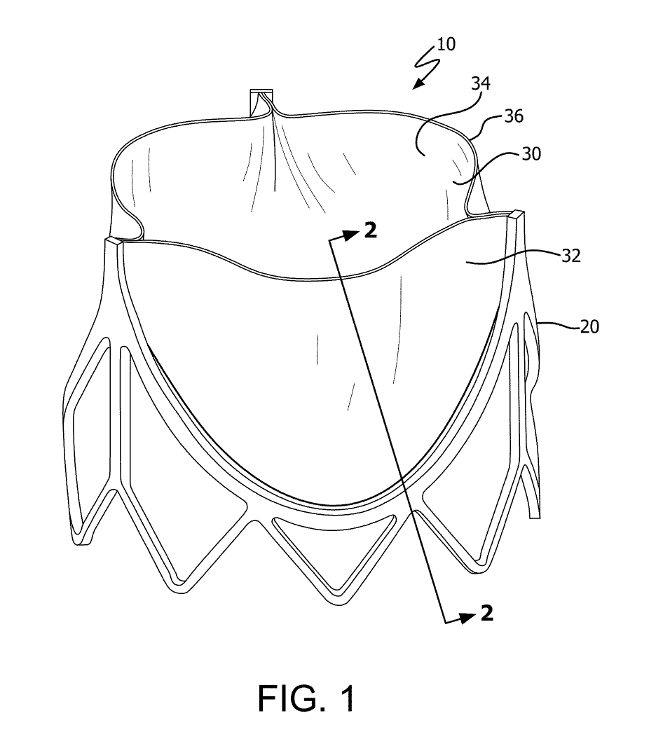

FIG. 1 is a perspective view of a prosthetic valve 10 in accordance with an embodiment. The prosthetic valve 10 comprises a frame 20 and leaflets 30. Each leaflet 30 has an inflow side 34 and an outflow side 32 and a free edge 36.

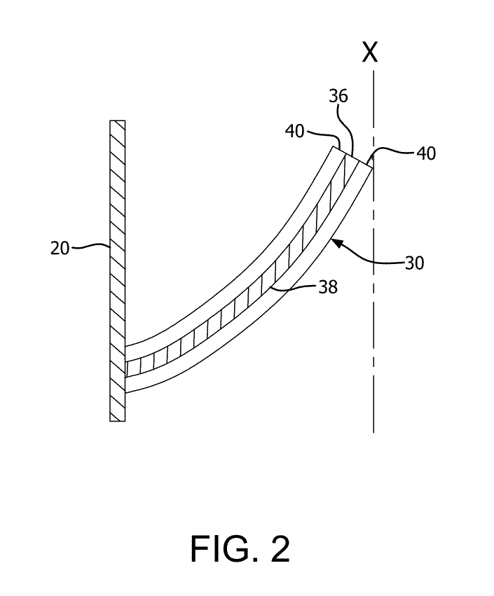

FIG. 2 is a cross sectional view of the prosthetic valve leaflet 30 coupled to the support structure 20 in accordance with the embodiment of FIG. 1 along outline 2-2. The leaflet 30 includes a composite material 38 and a TFE-PMVE copolymer coating 40 defining the inflow side 34 and outflow side 32.

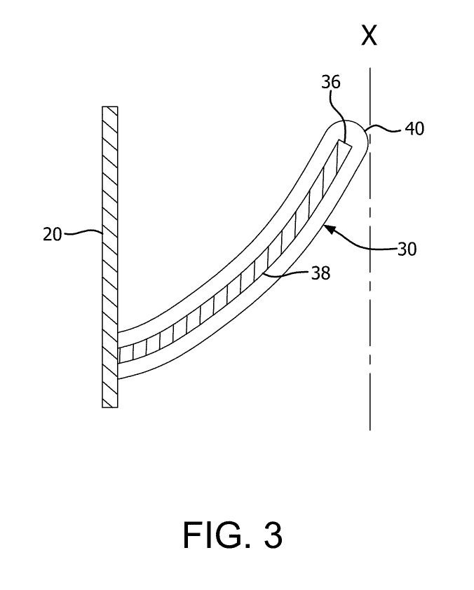

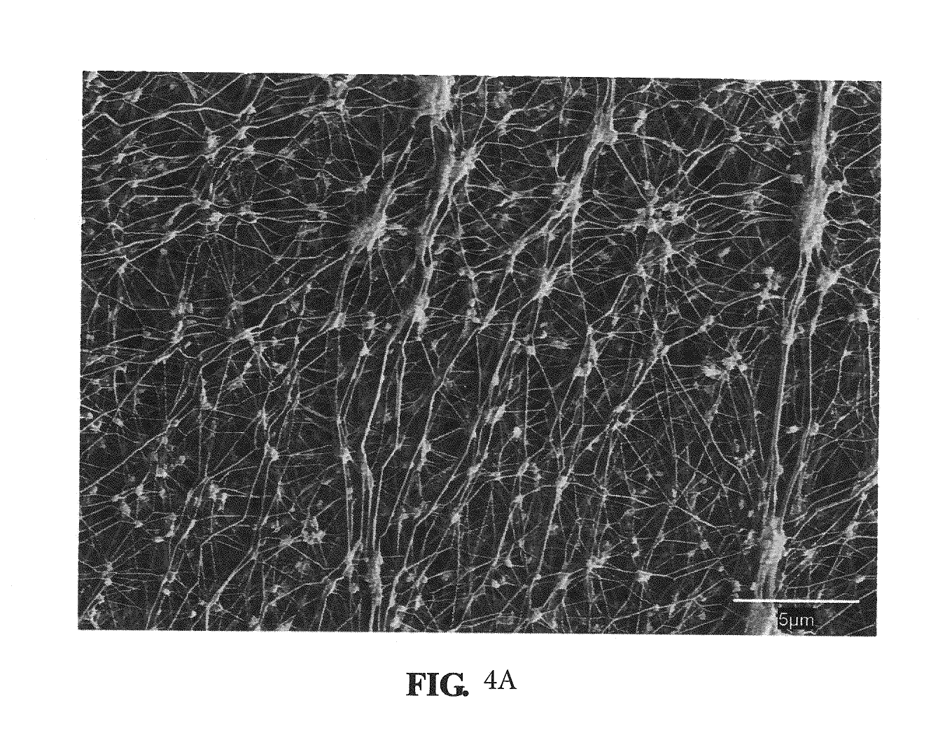

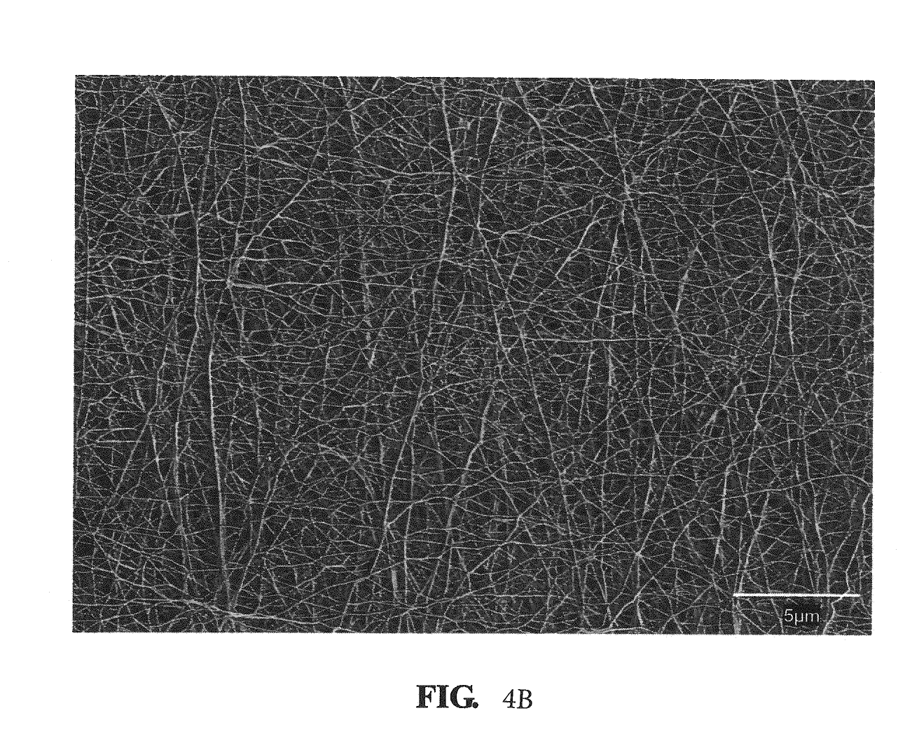

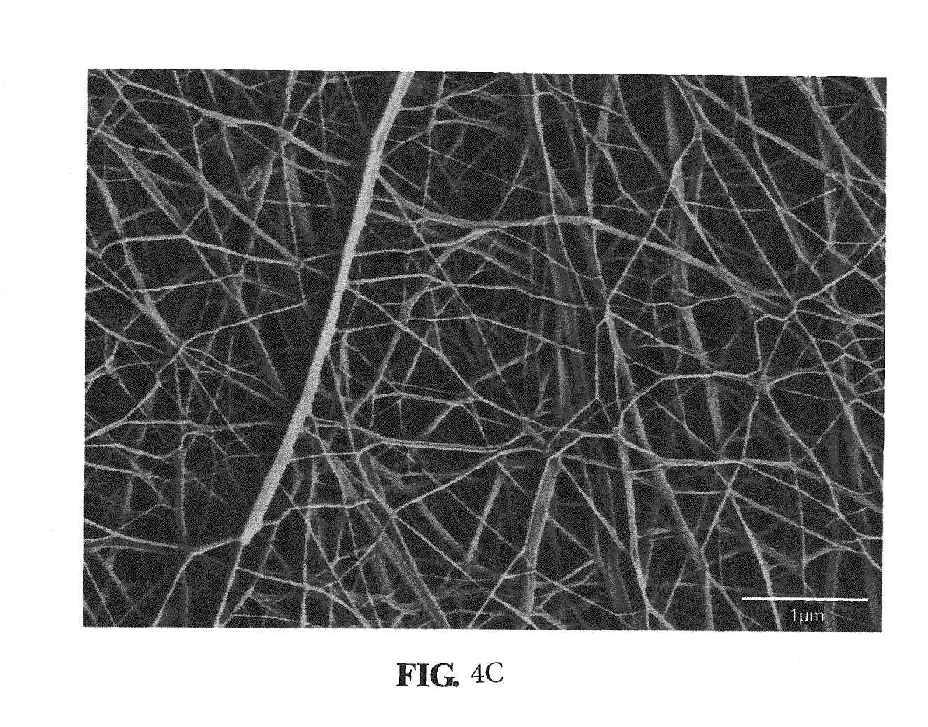

FIG. 3 is a cross sectional view of a prosthetic valve leaflet 30 in accordance with another embodiment substantially the same as the embodiment of FIG. 2 but additionally showing the TFE-PMVE copolymer coating 40 also on the free edge 36.

A TFE-PMVE copolymer coating 40 comprising from about 27 to about 32 weight percent perfluoromethyl vinyl ether and respectively from about 73 to about 68 weight percent tetrafluoroethylene to the blood-contacting surfaces of the composite material 38 results in a reduction of calcification under certain controlled laboratory conditions.

Further, a TFE-PMVE copolymer coating comprising from about 27 to about 32 weight percent perfluoromethyl vinyl ether and respectively from about 73 to about 68 weight percent tetrafluoroethylene to the surfaces of the leaflet and other valve components results in a reduction of the tackiness possibly found in a porous synthetic polymer membrane having been imbibed with certain TFE-PMVE copolymers, such as, but not limited to, certain of TFE-PMVE copolymer comprising from about 40 to about 80 weight percent perfluoromethyl vinyl ether and respectively from about 60 to about 20 weight percent tetrafluoroethylene. The corresponding tackiness is undesirable particularly with the handling characteristics of the prosthetic valve 10. Among other things, leaflets 30 having a tacky surface can result in a prosthetic valve wherein the leaflets 30 become adhered together when compressed into a pre-deployment configuration for transcatheter placement. In some embodiments there may be a continuous coating or layer of a TFE-PMVE copolymer comprising from about 27 to about 32 weight percent perfluoromethyl vinyl ether and respectively from about 73 to about 68 weight percent tetrafluoroethylene. In other embodiments there may be a discontinuous coating or layer or a combination of a continuous coating or layer on a portion and a discontinuous coating or layer on another portion. An example of a discontinuous layer or coating, such as a powder, comprises a TFE-PMVE copolymer comprising from about 27 to about 32 weight percent perfluoromethyl vinyl ether and respectively from about 73 to about 68 weight percent tetrafluoroethylene on surfaces of the leaflet and/or other valve components will result in a reduction of the tackiness found in a porous synthetic polymer membrane having been imbibed with certain TFE-PMVE copolymers.

In addition, it is appreciated that a discontinuous layer or coating, such as a powder, comprising a TFE-PMVE copolymer comprising from about 27 to about 32 weight percent perfluoromethyl vinyl ether and respectively from about 73 to about 68 weight percent tetrafluoroethylene on surfaces of the leaflet and other valve components will result in a reduction of the tackiness found in a porous synthetic polymer membrane having been imbibed with certain TFE-PMVE copolymers.

A coating of a TFE-PMVE copolymer comprising from about 27 to about 32 weight percent perfluoromethyl vinyl ether and from about 73 to about 68 weight percent tetrafluoroethylene to the blood-contacting surfaces of the composite material significantly increases the flexural durability of polymer prosthetic valve leaflets. Exemplary embodiments of the leaflets include a porous synthetic polymer membrane wherein elastomer of from about 40 and to about 80 weight percent perfluoromethyl vinyl ether and from about 60 and 20 weight percent tetrafluoroethylene or an elastomeric material of from about 33 to about 39 weight percent perfluoromethyl vinyl ether and from about 67 to about 61 weight percent tetrafluoroethylene, fills the pores of the porous synthetic polymer membrane, further including a layer of TFE-PMVE copolymer comprising from about 27 to about 32 weight percent perfluoromethyl vinyl ether and from about 73 to about 68 weight percent tetrafluoroethylene.

A leaflet material according to one embodiment includes an expanded fluoropolymer membrane and an elastomeric material, and further comprising a coating of TFE-PMVE copolymer comprising from about 27 to about 32 weight percent perfluoromethyl vinyl ether and from about 73 to about 68 weight percent tetrafluoroethylene. It should be readily appreciated that multiple types of fluoropolymer membranes and multiple types of elastomer and elastomeric materials can be combined while within the spirit of the present disclosure.

In some embodiments, the porous synthetic polymer membrane includes an expanded fluoropolymer material made from porous ePTFE membrane, for instance as generally described in U.S. Pat. No. 7,306,729. In some other embodiments, the porous synthetic polymer membrane includes a polyethylene material made from porous polyethylene membrane.

The expandable fluoropolymer, used to form the expanded fluoropolymer material described in embodiments, may comprise PTFE homopolymer. In alternative embodiments, blends of PTFE, expandable modified PTFE and/or expanded copolymers of PTFE may be used. Non-limiting examples of suitable fluoropolymer materials are described in, for example, U.S. Pat. No. 5,708,044, to Branca, U.S. Pat. No. 6,541,589, to Baillie, U.S. Pat. No. 7,531,611, to Sabol et al., U.S. patent application Ser. No. 11/906,877, to Ford, and U.S. patent application Ser. No. 12/410,050, to Xu et al.

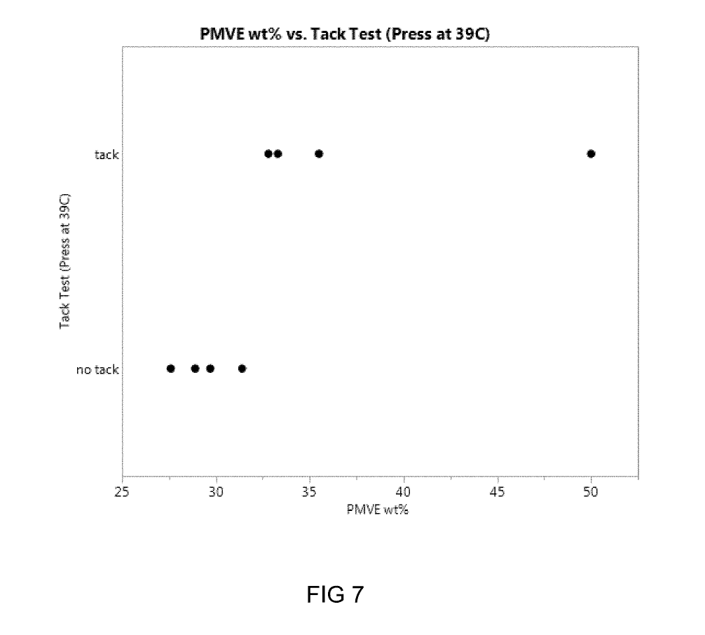

The expanded fluoropolymer membrane in accordance with some embodiments, may comprise any suitable microstructure for achieving the desired leaflet performance. In one embodiment, the expanded fluoropolymer may comprise a microstructure of nodes interconnected by fibrils, such as described in U.S. Pat. No. 3,953,566 to Gore. In one embodiment, the microstructure of an expanded fluoropolymer membrane comprises nodes interconnected by fibrils as shown in the scanning electron micrograph image in FIG. 7A. The fibrils extend from the nodes in a plurality of directions, and the membrane has a generally homogeneous structure. Membranes having this microstructure may exhibit a ratio of matrix tensile strength in two orthogonal directions of less than about 2, and, in another embodiment, less than about 1.5.

In another embodiment, the expanded fluoropolymer membrane may have a microstructure of substantially only fibrils, such as, for example, depicted in FIGS. 7B and 7C, as is generally taught by U.S. Pat. No. 7,306,729, to Bacino. FIG. 7C is a higher magnification of the expanded fluoropolymer membrane shown in FIG. 7B, and more clearly shows the homogeneous microstructure having substantially only fibrils. The expanded fluoropolymer membrane having substantially only fibrils as depicted in FIGS. 7B and 7C, may possess a high surface area, such as greater than about 20 m²/g, or greater than about 25 m²/g, and in some embodiments may provide a highly balanced strength material having a ratio of matrix tensile strengths in two orthogonal directions of less than about 2, and possibly less than about 1.5. It is anticipated that expanded fluoropolymer membrane may have a mean flow pore sizes of less than about 5 μm, less than about 1 μm, and less than about 0.10 μm, in accordance with embodiments.

The expanded fluoropolymer membrane in accordance with some embodiments may be tailored to have any suitable thickness and mass to achieve the desired leaflet performance. In some cases, it may be desirable to use a very thin expanded fluoropolymer membrane having a thickness less than about 65 μm, and in another embodiments, between 20 μm and 65 μm. In other embodiments, it may be desirable to use an expanded fluoropolymer membrane having a thickness greater than about 0.1 μm and less than about 20 μm. The expanded fluoropolymer membranes can possess a specific mass less than about 1 g/m² to greater than about 50 g/m².

Membranes comprising expanded fluoropolymer according to an embodiment can have matrix tensile strengths ranging from about 50 MPa to about 400 MPa or greater, based on a density of about 2.2 g/cm³ for PTFE.

Additional materials may be incorporated into the pores or within the material of the membranes or in between layers of the membranes to enhance desired properties of the leaflet. Composites according to one embodiment can include fluoropolymer membranes having thicknesses ranging from about 100 μm to less than about 0.3 μm.

Embodiments of expanded fluoropolymer membrane combined with TFE-PMVE copolymer that exhibits elastomer, elastomeric, and non-elastomer properties provides performance attributes required for use in high-cycle flexural implant applications, such as prosthetic heart valve leaflets, in at least several significant ways. For example, the addition of TFE-PMVE copolymer that exhibits elastomer, elastomeric, and non-elastomer properties improves the fatigue performance of the leaflet by eliminating or reducing the stiffening observed with ePTFE-only materials. In addition, it reduces the likelihood that the material will undergo permanent set deformation, such as wrinkling or creasing, that could result in compromised performance. In one embodiment of a composite, the TFE-PMVE copolymer that exhibits elastomer, elastomeric, or non-elastomer properties occupies substantially all of the pore volume or space within the porous structure of the expanded fluoropolymer membrane. In another embodiment of the composite, the TFE-PMVE copolymer that exhibits elastomer, elastomeric, or non-elastomer properties is present in substantially all of the pores of the at least one fluoropolymer membrane. Having TFE-PMVE copolymer that exhibits elastomer, elastomeric, or non-elastomer properties filling the pore volume or present in substantially all of the pores of the at least one fluoropolymer membrane reduces the space in which foreign materials can be undesirably incorporated into the composite. Further, a layer or coating of TFE-PMVE copolymer comprising from about 27 to about 32 weight percent perfluoromethyl vinyl ether and respectively from about 73 to about 68 weight percent tetrafluoroethylene significantly reduces the possibility of the pores of the porous structure of the expanded fluoropolymer membrane from opening up due, in part, to creep characteristics of an elastomer or elastomeric material in the pores of the expanded fluoropolymer membrane over time being exposed to closing pressures and high-cycle flexure.

An example of such foreign material entering into spaces that may open up in the composite material comprising a porous structure of the expanded fluoropolymer membrane having an elastomer or elastomeric material in the pores is calcium. If calcium becomes incorporated into the composite material, as used for example in a prosthetic heart valve leaflet, mechanical damage can occur during cycling, thus leading to the formation of holes in the leaflet and degradation in hemodynamics.

In one embodiment, the elastomer that is imbibed into the ePTFE membrane is a thermoplastic copolymer of tetrafluoroethylene (TFE) and perfluoromethyl vinyl ether (PMVE), such as described in U.S. Pat. No. 7,462,675. As discussed above, the elastomer is imbibed into the expanded fluoropolymer membrane such that the elastomer occupies substantially all of the void space or pores within the expanded fluoropolymer membrane. This filling of the pores of the expanded fluoropolymer membrane with elastomer can be performed by a variety of methods known to those skilled in the art.

In one embodiment, a method of filling the pores of the expanded fluoropolymer membrane includes the steps of dissolving the elastomer in a solvent suitable to create a solution with a viscosity and surface tension that is appropriate to partially or fully flow into the pores of the expanded fluoropolymer membrane and allow the solvent to evaporate, leaving the filler behind.

In another embodiment, a method of filling the pores of the expanded fluoropolymer membrane includes the steps of delivering the filler via a dispersion to partially or fully fill the pores of the expanded fluoropolymer membrane;

In another embodiment, a method of filling the pores of the expanded fluoropolymer membrane includes the steps of bringing the porous expanded fluoropolymer membrane into contact with a sheet of the elastomer or elastomeric material under conditions of heat and/or pressure that allow elastomer or elastomeric material to flow into the pores of the expanded fluoropolymer membrane.

In another embodiment, a method of filling the pores of the expanded fluoropolymer membrane includes the steps of polymerizing the elastomer within the pores of the expanded fluoropolymer membrane by first filling the pores with a prepolymer of the elastomer and then at least partially curing the elastomer.

A TFE-PMVE copolymer comprising from about 27 to about 32 weight percent perfluoromethyl vinyl ether and respectfully from about 73 to about 68 weight percent tetrafluoroethylene, for purposes of this disclosure, is considered not an elastomer or elastomeric material and will be referred to herein as “non-elastomeric TFE-PMVE copolymer”, which is an example of a “non-elastomeric material”. Being non-soluble, the non-elastomeric TFE-PMVE copolymer can be thermally formed, as with extrusion, into a sheet suitable for coupling to the fluoropolymer membrane.

In an embodiment, a method of coating a composite material, that is expanded fluoropolymer membrane imbibed with elastomer or elastomeric material, with a non-elastomeric TFE-PMVE copolymer having from about 27 to about 32 weight percent perfluoromethyl vinyl ether and respectively from about 73 to about 68 weight percent tetrafluoroethylene, includes the steps of bringing the composite material into contact with a sheet of the non-elastomeric TFE-PMVE copolymer under conditions of heat and/or pressure that allow the non-elastomeric TFE-PMVE copolymer to couple with the composite material. By way of example, but not limited thereto, a 1.5 μm thick layer of non-elastomeric TFE-PMVE copolymer, per the above, was coupled to an ePTFE membrane that was imbibed with elastomeric material comprising from about 33 to about 39 weight percent perfluoromethyl vinyl ether and respectively from about 72 to about 61 weight percent tetrafluoroethylene.

Other biocompatible polymers which may be suitable for use as the elastomer or elastomeric material may include, but not be limited to, the groups of urethanes, silicones (organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

In accordance with an embodiment, the composite material comprises an elastomeric material comprising the TFE-PMVE copolymer having from about 33 to about 39 weight percent perfluoromethyl vinyl ether and respectively from about 67 to about 61 weight percent tetrafluoroethylene imbibed into an ePTFE membrane. In an embodiment of the composite material, TFE-PMVE copolymer is present in the pores of an ePTFE membrane rendering the ePTFE impermeable. In accordance with another embodiment, the composite material comprises elastomer material comprising from about 40 to about 80 weight percent perfluoromethyl vinyl ether and respectively from about 60 to about 20 weight percent tetrafluoroethylene imbibed into a fluoropolymer membrane such as ePTFE or PTFE membrane.

In addition to expanded fluoropolymer membrane, other biocompatible synthetic polymer membranes, such as, but not limited to, expanded polymer membrane, may be suitable for use as a porous membrane. In accordance with an embodiment, microporous polyethylene is provided as a biocompatible porous polymer membrane as suitable for the particular purpose.

An embodiment of a microporous polyethylene membrane includes a sheet of material comprising substantially all fibers having a diameter of less than about 1 μm. In another embodiment of a microporous polyethylene membrane includes a sheet of non-woven material wherein substantially all fibers have a diameter of less than about 1 μm. In some cases, it may be desirable to use a very thin microporous polyethylene membrane having a thickness less than about 10.0 μm. In other embodiments, it may be desirable to use a microporous polyethylene membrane having a thickness less than about 0.6 μm.

It is appreciated that the structure of the microporous membranes disclosed in embodiments provided herein, may be differentiated from other structures such as fabrics, knits and fiber windings, by looking at the specific surface area of the material. Embodiments of microporous membranes suitable may include those having a specific surface area of greater than about 4.0 m²/cc. In accordance with other embodiments of microporous membranes provided herein have a specific surface area of greater than about 10.0 m²/cc. The embodiments provided herein appreciate that a membrane having a specific surface area of greater than about 4.0 to more than about 60 m²/cc provide a significant improvement to, at least, but not limited to, the durability and lifetime of the heart valve when used as leaflet material.

It is appreciated that microporous membranes disclosed in embodiments provided herein may alternatively be differentiated from other structures such as fabrics, knits and fiber windings, by looking at the fiber diameter of the material. Embodiments of microporous membranes provided herein contain a majority of fibers having a diameter that is less than about 1 μm. Other embodiments of microporous membranes provided herein contain a majority of fibers having a diameter that is less than about 0.1 μm. The embodiments provided herein recognize that a membrane comprising fibers the majority of which are less than about 1 to beyond less than about 0.1 μm provide a significant improvement to, at least, but not limited to, the durability and lifetime of the heart valve when used as leaflet material.

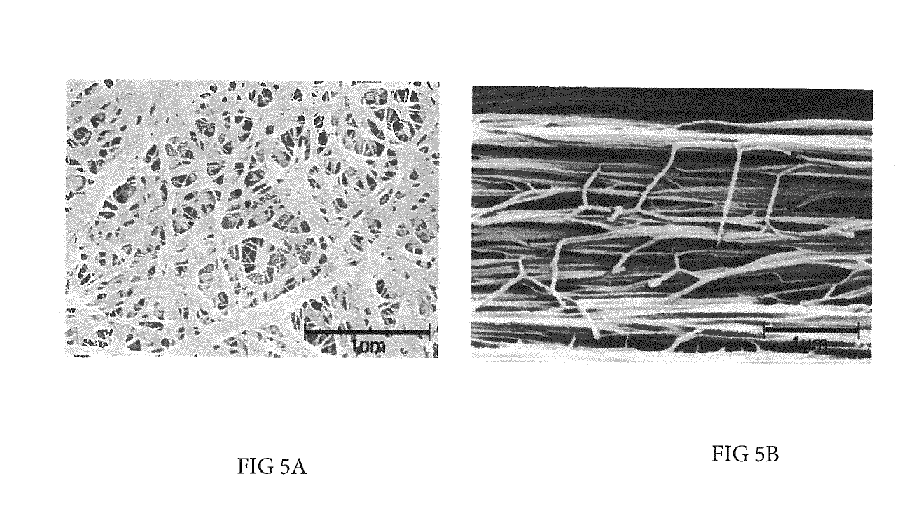

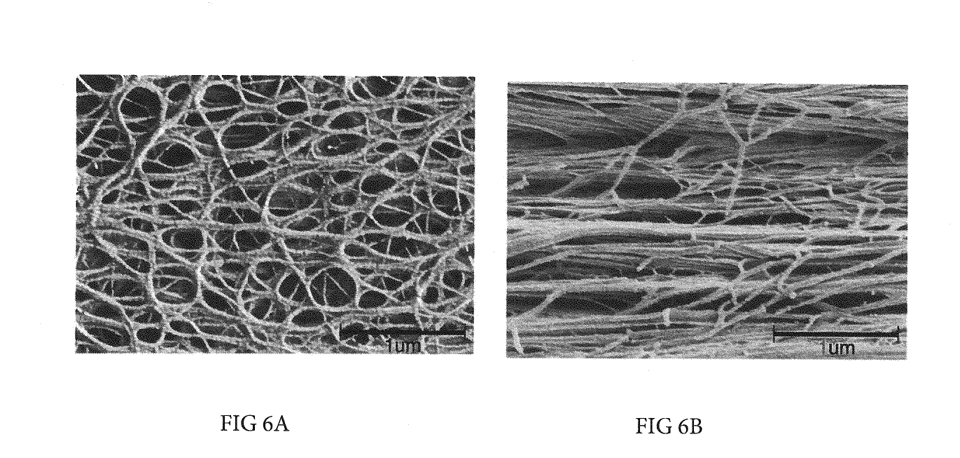

The microporous polymer membranes of embodiments may comprise any suitable microstructure and polymer for achieving the desired leaflet performance. In some embodiments, the microporous polymer membrane is porous polyethylene that has a microstructure of substantially only fibers, such as, for example, depicted in FIGS. 5A and 5B and FIGS. 6A and 6B. FIG. 5 shows a substantially homogeneous microstructure of the porous polyethylene membrane having substantially only fibers having a diameter of less than about 1 μm. The porous polyethylene membrane had a thickness of 0.010 mm, a porosity of 31.7%, a mass/area of 6.42 g/m², and a specific surface area of 28. 7 m²/cc.

FIGS. 6A and 6B, a surface and cross-sectional view, respectively, is the same porous polyethylene membrane shown in FIGS. 5A and 5B, a surface and cross-sectional view, respectively, that has been stretched in a process known in the art. The stretched polyethylene membrane retains a substantially homogeneous microstructure having substantially only fibers having a diameter of less than about 1 μm. The stretched polyethylene membrane has a thickness of 0.006 mm, a porosity of 44.3%, a mass/area of 3.14 g/m², and a specific surface area of 18.3 m²/cc. It is anticipated that microporous polyethylene membrane may have a mean flow pore sizes of less than about 5 μm, less than about 1 μm, and less than about 0.10 μm, in accordance with embodiments.

In addition to porous membrane, it is appreciated that non-porous materials may be coated with the non-elastomeric TFE-PMVE copolymer comprising from about 27 to about 32 weight percent perfluoromethyl vinyl ether and respectively from about 73 to about 68 weight percent tetrafluoroethylene suitable for a particular purpose. Among other things, it is appreciated that the non-elastomeric TFE-PMVE copolymer provides a non-tacky material that resists leaflet adhesion when the prosthetic valve is in the compressed state prior to transcatheter placement. It is appreciated that medical devices, such as, but not limited to, vascular grafts and prosthetic valve leaflets, provided with non-tacky surfaces have particular handling advantages over those having a tacky or sticky surface.

In accordance with some embodiments, prosthetic valve leaflets can comprise a single ply of a porous synthetic polymer membrane, that is, a single layer that is porous, wherein the pores contain an elastomer or elastomeric TFE/PMVE copolymer material rendering the single ply of a porous synthetic polymer membrane impermeable. The leaflet material comprising a single ply of a porous synthetic polymer membrane that contains an elastomer or elastomeric material rendering the single ply of a porous synthetic polymer membrane single layer impermeable, further coated with a layer of non-elastomeric TFE-PMVE copolymer, exhibits resistance to elastomer or elastomeric material creep under flexion so as to prevent surface porosity as evidenced in laboratory testing. Prevention of surface porosity is important to provide a surface resistant to calcification, among other benefits.

It is understood that the leaflet material provided by embodiments presented herein can be formed into leaflets to provide a structure that functions as a prosthetic valve. Such leaflets may further be attached to a frame by any suitable means, including sewing, adhesive, clips and other mechanical attachments. In accordance with an embodiment, the frame is selectively diametrically adjustable for endovascular delivery and deployment at a treatment site.

In accordance with embodiments, a prosthetic valve is provided that comprises a frame and a leaflet coupled to the frame. The leaflet comprises a composite having one ply of a porous synthetic polymer membrane imbibed with an elastomer or elastomeric material, and a coating of TFE-PMVE copolymer comprising from about 27 to about 32 weight percent perfluoromethyl vinyl ether and respectively from about 73 to about 68 weight percent tetrafluoroethylene. The single ply of a porous synthetic polymer membrane has a porous structure. The elastomer is present in the pores rendering the single ply of a porous synthetic polymer membrane impermeable. In accordance with embodiments, the layer of non-elastomeric TFE-PMVE copolymer comprising from about from about 27 to about 32 weight percent perfluoromethyl vinyl ether and respectively from about 73 to about 68 weight percent tetrafluoroethylene is coupled to the leaflet inflow side and the leaflet outflow side opposite the leaflet inflow side. In another embodiment, at least the leaflet free edge is also provided with the layer of the non-elastomeric TFE-PMVE copolymer. In another embodiment, the entire leaflet, including the leaflet inflow side and the leaflet outflow side opposite the inflow side, and the leaflet free edge therebetween is also provided with a layer of the non-elastomeric TFE-PMVE copolymer, whereby encapsulating the composite material. In the later embodiment, the non-elastomeric TFE-PMVE copolymer effectively contains the elastomer or elastomeric material of the composite material within the single ply of the porous synthetic polymer membrane, so as to prevent creep. Further, in accordance with embodiments, the non-elastomeric TFE-PMVE copolymer effectively provides the leaflet with a non-tacky property. According to an embodiment, the non-elastomeric TFE-PMVE copolymer is a coating having a thickness of 0.25 μm to 30 μm. In another embodiment, the non-elastomeric TFE-PMVE copolymer is a coating having a thickness of 0.5 μm to 15 μm. In other embodiments. In other embodiments, the thickness of the non-elastomeric TFE-PMVE copolymer coating is variable along the composite material. By way of example, the non-elastomeric TFE-PMVE copolymer coating may only be on a surface of the composite material that is expected to come into contact with another leaflet so as to prevent the two leaflets from sticking together when in contact. By way of another example, the thickness of the non-elastomeric TFE-PMVE copolymer coating may be different on the inflow side than on the outflow side to accommodate for anticipated stress on the leaflet, contact with other leaflets or itself, or to influence bending characteristics of the leaflet.

Tack Test

In accordance with embodiments, the leaflet passes a tack test as provided herein. The tack test assesses the resistance of a film, or leaflet comprising such film, to stick to another surface. In accordance with the test, a number of pairs of TFE-PMVE films, each member of the pair comprising similar weight percent of perfluoromethyl vinyl ether and respective weight percent tetrafluoroethylene, were provided and placed in direct contact with each other. The respective pair of TFE-PMVE films were then sandwiched between polyimide films and pressed in a Model M Carver press (Carver Laboratory Press, Wasbash Ind. USA) at 39° C., 200 psi for 15 minutes. After 15 minutes, the pairs of TFE-PMVE film were removed from the press and the polyimide films were removed. The pair of two TFE-PMVE films were then separated from each other, if there was no adherence between the two TFE-PMVE films and no force was required to separate the two TFE-PMVE films, the TFE-PMVE composition was determined to have “no tack”. A pair of two TFE-PMVE films that required force to separate the two TFE-PMVE films from each other were determined to have “tack”.

FIG. 7 is a graph of results of the tack test on various compositions of the TFE-PMVE films. It is noted that a pair of TFE-PMVE films having a weight percent perfluoromethyl vinyl ether that is greater than 27 presents a positive tack result. No tack is found for a TFE-PMVE composition having equal to or less than about 27 weight percent perfluoromethyl vinyl ether.

According to an embodiment (embodiment 1), a medical device includes a TFE-PMVE copolymer comprising from about 27 to about 32 weight percent perfluoromethyl vinyl ether and respectively from about 73 to about 68 weight percent tetrafluoroethylene.

According to another embodiment (embodiment 2), further to embodiment 1, the TFE-PMVE copolymer is coupled to a surface of the medical device.

According to another embodiment (embodiment 3), further to embodiment 1, the TFE-PMVE copolymer is a coating on at least a portion of the medical device.

According to another embodiment (embodiment 4), further to embodiment 1, the TFE-PMVE copolymer is a layer that is coupled to a surface of the medical device.

According to another embodiment (embodiment 5), further to embodiments 1-4, the medical device comprises a prosthetic valve leaflet, the leaflet having an inflow side and an outflow side opposite the inflow side, the TFE-PMVE copolymer being coupled to one or both of the inflow side and the outflow side.

According to another embodiment (embodiment 6), further to embodiments 1-4, the medical device comprises a synthetic polymer prosthetic valve leaflet, the leaflet having an inflow side and an outflow side opposite the inflow side, the TFE-PMVE copolymer being coupled to one or both of the inflow side and the outflow side.

According to another embodiment (embodiment 7), further to embodiments 1-4, the medical device comprises a synthetic polymer prosthetic valve leaflet, the leaflet having an inflow side and an outflow side opposite the inflow side, the TFE-PMVE copolymer being coupled to one or both of the inflow side and the outflow side rendering the respective side non-tacky per a tack test.

According to another embodiment (embodiment 8), further to embodiments 1-4, the medical device comprises a synthetic polymer prosthetic valve leaflet, the leaflet having an inflow side and an outflow side opposite the inflow side, the TFE-PMVE copolymer being coupled to the inflow side and the outflow side of the leaflet and a free edge defined by the inflow side and the outflow side.

According to another embodiment (embodiment 9), further to embodiments 1-4, the medical device comprises a synthetic polymer prosthetic valve leaflet, the leaflet having an inflow side and an outflow side opposite the inflow side defining an edge therebetween, the TFE-PMVE copolymer defining a coating encapsulating the inflow side, the outflow side and the edge.

According to another embodiment (embodiment 10), further to embodiments claims 5-9, the leaflet includes at least one ply of porous synthetic polymer membrane defining pores.

According to another embodiment (embodiment 11), further to embodiment 10, an elastomer or elastomeric material fills the pores of the porous synthetic polymer membrane defining a composite material, wherein the TFE-PMVE copolymer is a coating on the composite material.

According to another embodiment (embodiment 12), further to embodiment 11, the elastomer comprises from about 40 to about 80 weight percent perfluoromethyl vinyl ether and respectively from about 60 to about 20 weight percent tetrafluoroethylene.

According to another embodiment (embodiment 13), further to embodiment 11, the elastomeric material comprises from about 33 to about 39 weight percent perfluoromethyl vinyl ether and respectively from about 67 to about 61 weight percent tetrafluoroethylene.

According to another embodiment (embodiment 14), further to embodiments 10-13, the synthetic polymer membrane is an ePTFE membrane.

According to another embodiment (embodiment 15), further to embodiments 5-14, the leaflet passes a tack test.

According to another embodiment (embodiment 16), further to embodiments 5-15, the medical device further comprises a frame, wherein the leaflet is coupled to the frame and is movable between open and closed positions.

According to another embodiment (embodiment 17), further to embodiments 5-16, the TFE-PMVE copolymer comprising from about 27 to about 32 weight percent perfluoromethyl vinyl ether and respectively from about 73 to about 68 weight percent tetrafluoroethylene is melt processable.

According to another embodiment (embodiment 18), further to embodiments 1-17, the TFE-PMVE copolymer comprising from about 27 to about 32 weight percent perfluoromethyl vinyl ether and respectively from about 73 to about 68 weight percent tetrafluoroethylene is a coating having a thickness of 0.25 μm to 30 μm.

According to another embodiment (embodiment 19), further to embodiment 1-17, the TFE-PMVE copolymer comprising from about 27 to about 32 weight percent perfluoromethyl vinyl ether and respectively from about 73 to about 68 weight percent tetrafluoroethylene is a coating having a thickness of 0.5 μm to 4 μm.

According to another embodiment (embodiment 20), a medical device includes a TFE-PMVE copolymer comprising perfluoromethyl vinyl ether and tetrafluoroethylene wherein the medical device passes a tack test.

According to another embodiment (embodiment 21), further to embodiment 20, the TFE-PMVE copolymer comprises from about 27 to about 32 weight percent perfluoromethyl vinyl ether and respectively from about 73 to about 68 weight percent tetrafluoroethylene.

According to another embodiment (embodiment 22), further to embodiments 20-21, the medical device comprises a synthetic polymer prosthetic valve leaflet, the leaflet having an inflow side and an outflow side opposite the inflow side, the TFE-PMVE copolymer being coupled to one or both of the inflow side and the outflow side

According to another embodiment (embodiment 23), further to embodiments 20-21, the medical device comprises a synthetic polymer prosthetic valve leaflet, the leaflet having an inflow side and an outflow side opposite the inflow side, the TFE-PMVE copolymer being coupled to the inflow side and the outflow side of the leaflet and a free edge defined by the inflow side and the outflow side.

According to another embodiment (embodiment 24), further to embodiments 20-23, the leaflet includes at least one ply of porous synthetic polymer membrane.

According to another embodiment (embodiment 25), further to embodiment 24, an elastomer or elastomeric material fills the pores of the porous synthetic polymer membrane defining a composite material, wherein the TFE-PMVE copolymer is a coating on the composite material.

According to another embodiment (embodiment 26), further to embodiment 25, the elastomer comprises from about 40 to about 80 weight percent perfluoromethyl vinyl ether and respectively from about 60 to about 20 weight percent tetrafluoroethylene.

According to another embodiment (embodiment 27), further to embodiment 25, the elastomeric material comprises from about 34 to about 39 weight percent perfluoromethyl vinyl ether and respectively from about 66 to about 61 weight percent tetrafluoroethylene.

According to another embodiment (embodiment 28), further to embodiments 24-27, the synthetic polymer membrane is an ePTFE membrane.

According to another embodiment (embodiment 29), further to embodiments 22-28, the medical device further comprises a frame, wherein the leaflet is coupled to the frame and is movable between open and closed positions.

According to another embodiment (embodiment 30), further to embodiments 21-29, the TFE-PMVE copolymer comprising from about 27 to about 32 weight percent perfluoromethyl vinyl ether and respectively from about 73 to about 68 weight percent tetrafluoroethylene is melt processable.

According to another embodiment (embodiment 31), further to embodiments 22-30, the leaflet has a thickness of 20 μm to 65 μm.

According to another embodiment (embodiment 32), further to embodiments 20-30, the TFE-PMVE copolymer is a coating having a thickness of 0.25 μm to 30 μm.

According to another embodiment (embodiment 33), further to embodiments 20-30, the TFE-PMVE copolymer is a coating having a thickness of 0.5 μm to 4 μm.

According to another embodiment (embodiment 34), a synthetic prosthetic valve leaflet comprises a composite material including a porous synthetic polymer membrane defining pores and an elastomer or elastomeric material filling the pores and a TFE-PMVE copolymer comprising from about 27 to about 32 weight percent perfluoromethyl vinyl ether and respectively from about 73 to about 68 weight percent tetrafluoroethylene on at least a portion of the composite material.

According to another embodiment (embodiment 35), further to embodiment 34, the elastomer comprises from about 40 to about 80 weight percent perfluoromethyl vinyl ether and respectively from about 60 to about 20 weight percent tetrafluoroethylene.

According to another embodiment (embodiment 36), further to embodiment 34, the elastomeric material comprises from about 34 to about 39 weight percent perfluoromethyl vinyl ether and respectively from about 66 to about 61 weight percent tetrafluoroethylene.

According to another embodiment (embodiment 37), further to embodiments 34-36, the TFE-PMVE copolymer is coupled to an inflow side and an outflow side opposite the inflow side of the leaflet.

According to another embodiment (embodiment 38), further to embodiment 34-37, the TFE-PMVE copolymer renders the leaflet non-tacky wherein the leaflet passes a tack test.

According to another embodiment (embodiment 39), further to embodiments 34-38, the leaflet exhibits a ratio of tensile strength in two orthogonal directions of less than 2.

According to another embodiment (embodiment 40), further to embodiment 34-39, the porous synthetic polymer membrane is PTFE membrane.

According to another embodiment (embodiment 41), further to embodiment 40, the PTFE membrane is ePTFE membrane.

According to another embodiment (embodiment 42), further to embodiments 34-41, the TFE-PMVE copolymer is melt processable.

According to another embodiment (embodiment 43), further to embodiments 34-41, the TFE-PMVE copolymer is a coating having a thickness of 0.25 μm to 30 μm.

According to another embodiment (embodiment 44), further to embodiments 33-41, the TFE-PMVE copolymer is a coating having a thickness of 0.5 μm to 4 μm.

According to another embodiment (embodiment 45), further to embodiment 1, the medical device comprises a prosthetic valve leaflet, the leaflet includes at least one ply of porous synthetic polymer membrane defining pores imbibed with TFE-PMVE copolymer comprising from about 73 to about 68 weight percent tetrafluoroethylene and respectively about 27 to about 32 weight percent perfluoromethyl vinyl ether filling the pores.

According to another embodiment (embodiment 46), further to embodiment 45, wherein the leaflet is an expanded polytetrafluoroethylene (ePTFE) membrane.

According to another embodiment (embodiment 47), further to embodiment 46, the medical device comprises a prosthetic valve leaflet, the leaflet includes at least one ply of porous synthetic polymer membrane defining pores imbibed with TFE-PMVE copolymer comprising from about 73 to about 68 weight percent tetrafluoroethylene and respectively about 27 to about 32 weight percent perfluoromethyl vinyl ether filling the pores.

According to another embodiment (embodiment 48), further to embodiment 45, wherein the leaflet is an expanded polytetrafluoroethylene (ePTFE) membrane.

According to another embodiment (embodiment 49), further to embodiments 5-19, the leaflet has a thickness of 20 μm to 65 μm.

According to another embodiment (embodiment 50), further to embodiments 1-49, the TFE-PMVE copolymer comprising from about 27 to about 32 weight percent perfluoromethyl vinyl ether and respectively from about 73 to about 68 weight percent tetrafluoroethylene is a continuous coating, a discontinuous coating, or a combination of continuous and discontinuous coating.

Methods

According to another embodiment (embodiment 51), a method for reducing the tackiness of a medical device comprises coating at least a portion of the medical device with a TFE-PMVE copolymer comprising from about 27 to about 32 weight percent perfluoromethyl vinyl ether and respectively from about 73 to about 68 weight percent tetrafluoroethylene.

According to another embodiment (embodiment 52), further to embodiment 51, the medical device is a synthetic prosthetic valve leaflet.

According to another embodiment (embodiment 53), a method for reducing the calcification of a medical device comprises coating at least a portion of the medical device with a TFE-PMVE copolymer comprising from about 27 to about 32 weight percent perfluoromethyl vinyl ether and respectively from about 73 to about 68 weight percent tetrafluoroethylene.

According to another embodiment (embodiment 54), further to embodiment 53, the medical device is a synthetic prosthetic valve leaflet.

According to another embodiment (embodiment 55), a method for treating a human patient with a diagnosed condition or disease associated with valve insufficiency or valve failure of a native or prosthetic valve, the method comprising implanting a prosthetic valve comprising the leaflet of any of embodiments 34-50 at a location of the native or prosthetic valve.

According to another embodiment (embodiment 56), a method of making a prosthetic valve comprises obtaining a support structure that defines a base portion and a plurality of commissure posts, obtaining a plurality of leaflets of any of embodiments 34-50, and coupling the plurality of leaflets to the support structure by coupling an outer margin of each leaflet to the support structure with a free edge of each leaflet extending across an annular region defined by the support structure, coupling a respective cusp of each leaflet to the respective base portion and coupling a commissure region of each leaflet to respective commissure posts.

It will be apparent to those skilled in the art that various modifications and variations can be made in the present embodiments and examples of this disclosure without departing from the spirit or scope of the disclosure. Thus, it is intended that the disclosure is inclusive of modifications and variations provided they come within the scope of the appended claims and their equivalents.

What is claimed is:

1. A medical device, comprising:
a TFE-PMVE copolymer comprising from about 27 to about 32 weight percent perfluoromethyl vinyl ether and respectively from about 73 to about 68 weight percent tetrafluoroethylene.

2. The medical device of claim 1, wherein the TFE-PMVE copolymer is coupled to a surface of the medical device.

3. The medical device of claim 1, wherein the TFE-PMVE copolymer is a coating on at least a portion of the medical device.

4. The medical device of claim 1, wherein the TFE-PMVE copolymer is a layer that is coupled to a surface of the medical device.

5. The medical device of claim 1, wherein the medical device comprises a prosthetic valve leaflet, the leaflet includes at least one ply of porous synthetic polymer membrane defining pores imbibed with the TFE-PMVE copolymer comprising from about 27 to about 32 weight percent perfluoromethyl vinyl ether and respectively about 73 to about 68 weight percent tetrafluoroethylene filling the pores.

6. The medical device of claim 5, wherein the porous synthetic polymer membrane is an expanded polytetrafluoroethylene (ePTFE) membrane.

7. The medical device of claim 6, wherein the medical device comprises a prosthetic valve leaflet, the leaflet has a thickness of 20 μm to 65 μm.

8. The medical device of claim 1, wherein the medical device comprises a synthetic polymer prosthetic valve leaflet, the leaflet having an inflow side and an outflow side opposite the inflow side, the TFE-PMVE copolymer being coupled to one or both of the inflow side and the outflow side.

9. The medical device of claim 1, wherein the medical device comprises a synthetic polymer prosthetic valve leaflet, the leaflet having an inflow side and an outflow side opposite the inflow side, the TFE-PMVE copolymer being coupled to one or both of the inflow side and the outflow side rendering the respective side non-tacky per a tack test.

10. The medical device of claim 1, wherein the medical device comprises a synthetic polymer prosthetic valve leaflet, the leaflet having an inflow side and an outflow side opposite the inflow side, the TFE-PMVE copolymer being coupled to the inflow side and the outflow side of the leaflet and a free edge defined by the inflow side and the outflow side.

11. The medical device of claim 1, wherein the medical device comprises a synthetic polymer prosthetic valve leaflet, the leaflet having an inflow side and an outflow side opposite the inflow side defining an edge therebetween, the TFE-PMVE copolymer defining a coating encapsulating the inflow side, the outflow side and the edge.

12. The medical device of claim 7, wherein the leaflet includes at least one ply of porous synthetic polymer membrane defining pores.

13. The medical device of claim 12, wherein an elastomer or elastomeric material fills the pores of the porous synthetic polymer membrane defining a composite material, wherein the TFE-PMVE copolymer is a coating on the composite material.

14. The medical device of claim 13, wherein the leaflet has a thickness of 20 μm to 65 μm.

15. The medical device of claim 13, wherein the elastomer comprises from about 40 to about 80 weight percent perfluoromethyl vinyl ether and respectively from about 60 to about 20 weight percent tetrafluoroethylene.

16. The medical device of claim 13, wherein the elastomeric material comprises from about 33 to about 39 weight percent perfluoromethyl vinyl ether and respectively from about 67 to about 61 weight percent tetrafluoroethylene.

17. The medical device of claim 13, wherein the synthetic polymer membrane is an ePTFE membrane.

18. The medical device of claim 17, wherein the leaflet passes a tack test.

19. The medical device of claim 17, wherein the medical device further comprises a frame, wherein the leaflet is coupled to the frame and is movable between open and closed positions.

20. The medical device of claim 1, wherein the TFE-PMVE copolymer comprising from about 27 to about 32 weight percent perfluoromethyl vinyl ether and respectively from about 73 to about 68 weight percent tetrafluoroethylene is a coating having a thickness of 0.25 μm to 30 μm.

21. A medical device, comprising:
a TFE-PMVE copolymer comprising perfluoromethyl vinyl ether and tetrafluoroethylene wherein the medical device passes a tack test.

22. The medical device of claim 21, wherein the TFE-PMVE copolymer comprises from about 27 to about 32 weight percent perfluoromethyl vinyl ether and respectively from about 73 to about 68 weight percent tetrafluoroethylene.

23. A synthetic prosthetic valve leaflet, comprising:
a composite material including a porous synthetic polymer membrane defining pores and an elastomer or elastomeric material or non-elastomeric material filling the pores; and
a TFE-PMVE copolymer comprising from about 27 to about 32 weight percent perfluoromethyl vinyl ether and respectively from about 73 to about 68 weight percent tetrafluoroethylene on at least a portion of the composite material.

24. The synthetic prosthetic valve leaflet of claim 23, wherein the elastomer comprises from about 40 to about 80 weight percent perfluoromethyl vinyl ether and respectively from about 60 to about 20 weight percent tetrafluoroethylene.

25. The synthetic prosthetic valve leaflet of claim 23, wherein the elastomeric material comprises from about 34 to about 39 weight percent perfluoromethyl vinyl ether and respectively from about 66 to about 61 weight percent tetrafluoroethylene.

26. The synthetic prosthetic valve leaflet claim 23, wherein the non-elastomeric material is TFE-PMVE copolymer comprising from about 27 to about 32 weight percent perfluoromethyl vinyl ether and respectively from about 73 to about 68 weight percent tetrafluoroethylene filling the pores.

27. The synthetic prosthetic valve leaflet of claim 26, wherein the leaflet is an expanded polytetrafluoroethylene (ePTFE) membrane.

28. The synthetic prosthetic valve leaflet of claim 23, wherein the TFE-PMVE copolymer is coupled to an inflow side and an outflow side opposite the inflow side of the leaflet.

29. The synthetic prosthetic valve leaflet of claim 23, wherein TFE-PMVE copolymer renders the leaflet non-tacky wherein the leaflet passes a tack test.

30. The synthetic prosthetic valve leaflet of claim 23, wherein the leaflet exhibits a ratio of tensile strength in two orthogonal directions of less than 2.

31. The synthetic prosthetic valve leaflet of claim 23, wherein the porous synthetic polymer membrane is PTFE membrane.

32. The synthetic prosthetic valve leaflet of claim 31, wherein the PTFE membrane is ePTFE membrane.

33. The synthetic prosthetic valve leaflet of claim 32, wherein the TFE-PMVE copolymer is a coating having a thickness of 0.25 μm to 30 μm.

34. The synthetic prosthetic valve leaflet of claim 32, wherein the TFE-PMVE copolymer is a coating having a thickness of 0.5 μm to 4 μm.

35. A method for reducing the tackiness of a medical device, comprising:
coating at least a portion of the medical device with a TFE-PMVE copolymer comprising from about 27 to about 32 weight percent perfluoromethyl vinyl ether and respectively from about 73 to about 68 weight percent tetrafluoroethylene.

36. The method of claim 35 wherein the medical device is a synthetic prosthetic valve leaflet.

37. A method for reducing calcification of a medical device, comprising:
coating at least a portion of the medical device with a TFE-PMVE copolymer comprising from about 27 to about 32 weight percent perfluoromethyl vinyl ether and respectively from about 73 to about 68 weight percent tetrafluoroethylene.

38. The method of claim 37 wherein the medical device is a synthetic prosthetic valve leaflet.

39. A method for treating a human patient with a diagnosed condition or disease associated with valve insufficiency or valve failure of a native or prosthetic valve, the method comprising implanting a prosthetic valve at a location of the native or prosthetic valve, the prosthetic valve having leaflets that comprise a composite material including a porous synthetic polymer membrane defining pores and an elastomer or elastomeric material filling the pores, and a TFE-PMVE copolymer comprising from about 27 to about 32 weight percent perfluoromethyl vinyl ether and respectively from about 73 to about 68 weight percent tetrafluoroethylene on at least a portion of the composite material.

40. A method of making a prosthetic valve, comprising:
obtaining a support structure that defines a base portion and a plurality of commissure posts;
obtaining a plurality of leaflets that comprise a composite material including a porous synthetic polymer membrane defining pores and an elastomer or elastomeric material filling the pores, and a TFE-PMVE copolymer comprising from about 27 to about 32 weight percent perfluoromethyl vinyl ether and respectively from about 73 to about 68 weight percent tetrafluoroethylene on at least a portion of the composite material; and
coupling the plurality of leaflets to the support structure by coupling an outer margin of each leaflet to the support structure with a free edge of each leaflet extending across an annular region defined by the support structure, coupling a respective cusp of each leaflet to the respective base portion and coupling a commissure region of each leaflet to respective commissure posts.
